(19) United States
(12) Patent Application Publication
(10) Pub. No.: US 2018/0290961 A1
(43) Pub. Date: Oct. 11, 2018

(54) METHOD AND SYSTEM FOR OBTAINING A CARBOXYLIC ACID WHICH IS PRODUCED IN A FERMENTATION PROCESS

(71) Applicants: THYSSENKRUPP INDUSTRIAL SOLUTIONS AG, Essen (DE); thyssenkrupp AG, Essen (DE)

(72) Inventors: Joachim SCHÜTZE, Torgau (DE); Gerd BRAUN, Overath (DE)

(73) Assignees: THYSSENKRUPP INDUSTRIAL SOLUTIONS AG, Essen (DE); thyssenkrupp AG, Essen (DE)

(21) Appl. No.: 15/754,371

(22) PCT Filed: Aug. 26, 2016
(86) PCT No.: PCT/EP2016/070144

§ 371 Date: Feb. 22, 2018

(30) Foreign Application Priority Data
Sep. 2, 2015 (DE) 10 2015 216 815.8

(51) Int. Cl. C07C 51/43; C07C 51/47; B01D 61/02; B01D 61/14; B01D 61/58; B01D 9/00

(52) U.S. Cl. CPC C07C 51/43; C07C 51/47; B01D 61/022; B01D 61/025; B01D 61/027; B01D 61/145; B01D 2317/025; B01D 9/0013; B01D 2311/06; B01D 2311/2642; B01D 2311/18; B01D 2311/106; B01D 61/58

(57) ABSTRACT

A plant and a process may be utilized to isolate a carboxylic acid from a fermentation broth. The process may involve separating a biomass from the fermentation broth containing a salt of the carboxylic acid to produce a low-biomass solution. The separation of the biomass may be performed in a first step by centrifugation, separation, precoat filtration, or microfiltration, and in a second step by ultrafiltration. The process may further involve concentrating the salt of the carboxylic acid in the low-biomass solution. The concentrated solution may then be acidified. Further, precipitation of the carboxylic acid may be obtained by acidification.”

### TECHNICAL FIELD

The present invention relates to a process for isolating a carboxylic acid prepared in a fermentation process and a plant for carrying out the process.

### TECHNICAL BACKGROUND

Succinic acid is an important building block for the chemical industry, with an annual demand of about 15000 metric tons. It is used, for example, for the production of plastics.

An important factor for industrial utilization of succinic acids which are present in a mixture of materials (for example the carbohydrate-containing substrates produced by fermentation or synthesis) is the economics and efficiency of the purification, concentration and preparation in pure form of the corresponding acid. These considerations of course also apply to other carboxylic acids.

WO 2013/120924 A2 describes a process for the biotechnological preparation of fumaric acid by fermentation of a yeast strain, in which fumaric acid is obtained as ammonium fumarate in a concentration of from 20 to 50 g/l.

WO 2014/106532 A2 describes a process for purifying carboxylic acids from fermentation broths, in which a fermentation broth containing ammonium carboxylate salts is purified. The biomass is firstly separated off from the fermentation broth. The ammonium carboxylate salt contained in the biomass-free solution is subsequently converted by acidification into carboxylic acid and subjected to simulated moving bed (SMB) chromatography. The carboxylic acid solution obtained is then subjected to a further purification step, concentrated and passed to a crystallization stage for isolating the carboxylic acid. A further work-up process is described in DE 10 2010 025 167.

A disadvantage is the high technical outlay resulting from such processes, in particular due to the use of SMB chromatography.

The publication DE 10 2013 225 215 A1 describes a process for preparing and isolating carboxylic esters, in which esterification of at least one free carboxylic acid is carried out by addition of at least one alcohol.

The publication DE 689 20 520 T2 describes a process for separating keto-2L-gulonic acid from fermentation broth by crystallization, in which a filtration or centrifugation using a flocculant is carried out.

### DESCRIPTION OF THE INVENTION

The invention is based on the observation that sparingly soluble carboxylic acids such as fumaric acid and adipic acid cannot be worked up satisfactorily using the known purification methods. For example, it was found that fermentatively prepared fumaric acid precipitates at concentrations above about 5 g/l after acidification. In order to compensate for the resulting loss of product, alternative purification methods are necessary.

It is therefore an object of the invention to provide a process by means of which the disadvantages of the prior art can at least partially be avoided. In particular, a carboxylic acid should be able to be prepared in high yield.

This object is achieved by a process for isolating a carboxylic acid from a fermentation broth having the features of claim 1 and a plant having the features of claim 14. The further dependent claims indicate advantageous embodiments.

According to the invention, the process comprises the following steps:
a) Separation of the biomass from the fermentation broth containing a salt of the carboxylic acid to produce a low-biomass solution;
b) Concentration of the salt of the carboxylic acid in the low-biomass solution;
c) Acidification of the concentrated solution; and
d) Precipitation of the carboxylic acid obtained by acidification.

It has been found that the step of acidification does not, as is customary, have to be carried out between the removal of the biomass and the further work-up or purification, but can instead be carried out after the work-up or purification and concentration. Apart from finding a further way of carrying out the process, this has the advantage that sparingly soluble carboxylic acids can be obtained without being withdrawn from the process by undesired precipitation or without blocking membrane surfaces in subsequent steps. Accordingly, the process procedure disclosed here allows the use of highly concentrated solutions since the salt of the carboxylic acid (carboxylic acid salt) usually has a higher solubility than the carboxylic acid. For example, ammonium succinate (solubility about 750 g/l) can be concentrated to an about ten times greater concentration than succinic acid (solubility about 75 g/l).

This recognition can in turn be utilized for making the dimensions of the plant required for carrying out the process of the invention small and thus allow the economical recovery of very sparingly soluble and salt-forming carboxylic acids (solubility in water max. 15 g/l at 20° C.), e.g. adipic acid, aspartic acid, fumaric acid, glutamic acid, salicylic acid and tyrosine, and also sparingly soluble carboxylic acids (solubility max. 50 g/l at 20° C.), e.g. histidine, isoleucine, leucine, phenylalanine and methionine, on an industrial scale.

A further advantage of the process of the invention is the possibility of carrying out the concentration step by means of reverse osmosis and/or nanofiltration and/or membrane distillation. In known processes, the osmotic pressure of the acidified solution, made up of the osmotic pressure of the carboxylic acid and the osmotic pressure of the salt solution (for example ammonium sulfate solution), is too great for such a concentration step to be carried out using membrane processes. For this reason, concentration of the solution can be carried out only by thermal methods (e.g. evaporation). The present invention thus also solves this problem since the lower osmotic pressure of the unacidified solution makes it possible to integrate energy-efficient concentration techniques such as reverse osmosis and/or nanofiltration and/or membrane distillation into the process.

The carboxylic acid is preferably selected from the group consisting of fumaric acid, succinic acid, adipic acid, itaconic acid, threonine, methionine, aspartic acid, glutamic acid, oxalic acid, asparagine, glutamine, histidine, isoleucine, leucine, phenylalanine, tryptophan, tyrosine, valine and mixtures thereof. Preference is given to the carboxylic acid being succinic acid and the carboxylic acid salt being ammonium succinate.

1. Removal of the Biomass

In step a), the biomass produced in the fermentation preceding the process is separated from the fermentation broth. In the fermentation, the cultivated microorganism forms the carboxylic acid and releases this into the fermentation broth. In order to counter the decrease in the pH, substances to adjust the pH, for example ammonia, are usually added. As a result, the carboxylic acid is present as its salt, for example as ammonium salt of the carboxylic acid. The fermentation broth usually comprises not only the carboxylic acid salt and biomass but also metabolites of the microorganisms cultivated in the fermentation and residues of the nutrient solution(s). After removal of the biomass, a low-biomass, ideally substantially biomass-free, fermentation broth containing the salt of the carboxylic acid to be isolated is present.

In a preferred embodiment, the removal of the biomass in a first step is carried out by means of centrifugation, separation, precoat filtration and/or microfiltration and in a second step by means of ultrafiltration. The second step separates, for example, residual biomass, insoluble solids and relatively high molecular weight compounds from the fermentation broth. Membranes having a separation limit of from 5 to 20 kDa have been found to be useful for this purpose.

2. Purification

In a preferred embodiment of the invention, purification of the low-biomass or concentrated solution by nanofiltration, cation exchange, anion exchange and/or activated carbon purification is carried out before step c). When pure starting materials are used, as in the treatment of synthesis products, it is usual not to carry out purification as described in this chapter.

The purification step is preferably carried out between step a) and b).

The way in which the purification is carried out usually depends on the solution to be purified and the required quality of the carboxylic acid (in particular in respect of the purity of the crystals) and the purification steps are optionally combined. Suitable combinations are, in particular: nanofiltration, cation exchange, anion exchange and activated carbon purification; nanofiltration, cation exchange and anion exchange; nanofiltration and cation exchange; nanofiltration, cation exchange and activated carbon purification; nanofiltration and activated carbon purification.

For example, a nanofiltration can be combined with cation exchange, anion exchange and activated carbon purification in order to attain a purity of ≥99%. As an alternative, a nanofiltration can be combined with cation exchange and anion exchange for this purpose.

To attain a purity of at least 90% (i.e. technical-grade purity), it is possible, for example, to combine a nanofiltration with cation exchange; cation exchange with anion exchange and activated carbon purification; or to combine cation exchange with activated carbon purification. To attain a purity of at least 90%, a nanofiltration can also be carried out.

In one embodiment of the invention, the nanofiltration is carried out at a separation limit of from 100 Da to 400 Da, preferably from 100 to 200 Da.

3. Concentration

In step b), the carboxylic acid salt, for example the ammonium salt of the carboxylic acid, is concentrated. This has the above-described advantage that the subsequent process steps can be carried out in smaller apparatuses than when a concentration step is omitted or is arranged further downstream in the process. Instead of using costly thermal processes, e.g. evaporation, concentration of the carboxylic acid salt solution can thus be carried out effectively using membrane processes.

In a preferred embodiment, concentration is carried out by means of a single-stage, two-stage or multistage membrane process. Suitable membrane processes are nanofiltration, reverse osmosis, high-pressure reverse osmosis, membrane distillation, with any combinations of the abovementioned processes also being able to be carried out. The advantage of carrying out combined membrane processes is that it is possible to obtain solutions having an osmotic pressure above 35 bar without precipitation of the carboxylic acid salt in the membrane plant occurring.

The temperature prevailing in the concentration step is selected as a function of the solubility of the carboxylic acid salt to be concentrated and is usually from 30° C. to 90° C. The final concentration of the carboxylic acid salt is dependent, in particular, on its solubility and is typically selected so that supersaturation of the solution and the associated crystallization occurs on cooling to from 10° C. to 40° C., in particular to from 25° C. to 30° C. Cooling to about 25° C. has the advantage that it can be achieved in a simple manner by means of cooling water. As a guideline, a final concentration of from 5 to 50% by weight, preferably from 5 to 20 to 25% by weight, may be indicated. This value is particularly preferred for the salt of succinic acid (e.g. ammonium succinate). For the subsequent precipitation to proceed efficiently, the concentration obtained in the concentration step b) should be above the solubility concentration of the carboxylic acid. Ideally, the salt of the carboxylic acid has a two-fold, preferably five-fold, in particular ten-fold, greater solubility in the concentrated solution than the carboxylic acid.

In one embodiment, the membrane process is a reverse osmosis and is carried out in two stages. The permeate from the first reverse osmosis stage is fed to the second reverse osmosis stage and the permeate from the second reverse osmosis stage is fed to an upstream process step. For example, the permeate can be fed to the purification (here, in particular, as solution for the diafiltration in a first nanofiltration) or the fermentation (here, in particular, as starting solution for the carbon source, nutrient salts or nutrients for the fermentation). The concentrate from the first reverse osmosis stage is fed to the next process step c). The concentrate from the second stage is usually mixed into the feed stream to the first stage.

In a further embodiment, the membrane process is a membrane distillation and the distillate from the membrane distillation is fed to an upstream process step. For example, the distillate can be fed to the purification (here, in particular, as solution for the diafiltration in a first nanofiltration) or the fermentation (here, in particular, as starting solution for the carbon source, nutrient salts or nutrients for the fermentation).

The membrane distillation is preferably carried out at temperatures just below the solubility limit of the carboxylic acid used, preferably in the range from 40° C. to 80° C. If the carboxylic acid is succinic acid, the concentration step is preferably carried out at temperatures in the range from 40° C. to 60° C. The membrane distillation has the advantage that solutions having a very high osmotic pressure (>140 bar) can be concentrated. Up to 140 bar, on the other hand, high-pressure reverse osmosis can be employed.

After the concentration step, a concentrated solution which, depending on the purification carried out, consists of a concentrated aqueous solution of the carboxylic acid salt and 10%, preferably 1%, of impurities is obtained. The pH of this solution is usually from 5.0 to 8.0, in particular from 6.0 to 7.0.

4. Acidification

In step c), the concentrated solution obtained by means of step b) is acidified. For example, a mineral acid is added in order to convert the carboxylic acid salt into the carboxylic acid. This is typically achieved by setting the pH to a desired value.

In a preferred embodiment, the acidification is carried out using a sulfuric acid. The pH is typically set to from 1.8 to 3.0 and in particular from 2.0 to 2.5.

The carboxylic acid preferably has a solubility in the acidified solution of from 60 to 110 g/l as in the case of succinic acid and itaconic acid, a solubility of from 30 to 55 g/l as in the case of methionine and histidine; or a solubility of from 5 to 25 g/l as in the case of adipic acid and fumaric acid.

5. Precipitation

Owing to the usually lower solubility of the carboxylic acid compared to the salt of the carboxylic acid, a first precipitation of the carboxylic acid can be carried out in step d). If the carboxylic acid precipitates virtually completely, further precipitation measures are optional. In all other cases, a precipitation (optionally additional precipitation) of the carboxylic acid is carried out. The precipitation is ideally a crystallization, for example a crystallization comprising cooling crystallization, and isolation of the crystallized carboxylic acid. The crystallization is preferably carried out as a fractional crystallization. The solubility of the inorganic salts formed by addition of the acid, for example ammonium sulfate, should be higher than the solubility of the carboxylic acid so that the carboxylic acid is separated off by precipitation from the dissolved inorganic salt. The carboxylic acid, which has ideally been precipitated in crystal form, can subsequently be isolated.

In order to isolate the carboxylic acid in crystalline form, it can be cooled and crystallized by means of a cooling crystallizer, preferably a contact crystallizer. In cooling crystallization, the crystals are precipitated in the mother liquor, with the mother liquor which has been separated from the crystals preferably being recirculated to the process. Thus, in a preferred embodiment, the mother liquor remaining after isolation of the crystallized carboxylic acid is, after the concentration of the carboxylic acid present therein has been increased, fed to the cooling crystallization in order to increase the yield. This can, for example, be effected by the mother liquor being taken off and subsequently fed to the concentration step (e.g. the reverse osmosis) or the purification (e.g. the nanofiltration); or subsequently being fed to a cooling crystallization, for example in a small contact crystallizer using cold water or cooling brine as cooling medium.

The remaining mother liquor, which contains essentially only ammonium sulfate solution, can be worked up by multistage evaporation and crystallization in order to isolate ammonium sulfate.

If the cooling crystallization is carried out in two stages, the cooling in the first stage can thus be carried out using the carboxylic acid solution taken off from the cooling crystallizer and in the second stage can thus be carried out using cold water or cooling brine introduced from the outside.

In an example, step b) is carried out by means of a membrane process and step d) is carried out by means of a cooling crystallization. In a preferred way of carrying out the process, the concentrate from the membrane process is subjected to regenerative heat exchange in a heat exchanger and heat exchange is effected using a mother liquor taken off from the cooling crystallization. The concentrate is preferably cooled to a temperature of from 30° C. to 40° C. and subsequently fed to the cooling crystallization. Here, it can be provided for the mother liquor which has been heated up in the heat exchanger to be purified by means of nanofiltration in a further step. The concentrate, namely the inorganic salt solution, can be fed to a conventional evaporation, while the permeate, namely the carboxylic acid solution, is recirculated to the cooling crystallization.

6. Energy and Materials Regeneration

The process of the invention can be made efficient by the concentration step (step b) and/or the purification being carried out with at least partial recovery of the energy consumed by the concentration and/or fine purification step.

For example, the reverse osmosis (see step b) can be provided with a pressure exchanger for recovering energy. In addition, the vapor obtained from the thermal concentration of the inorganic salt solution can be passed to thermal utilization in the membrane distillation.

For materials regeneration, the discharge streams obtained in the concentration step and/or the purification can be fed to the process upstream and/or to an esterification, preferably by means of ethanol. For example, the permeate from the first reverse osmosis stage can be fed to a second reverse osmosis stage and the permeate from the second reverse osmosis stage can be fed to an upstream process step. Furthermore, the distillate from the membrane distillation can be fed to an upstream process step. Details of this have been described above.

7. Plant Configured for Carrying Out the Process

The invention further provides a plant equipped for carrying out the process described here. The plant configured for isolating a carboxylic acid from a fermentation broth containing the salt of the carboxylic acid comprises, according to the invention:
a) a separation unit for separating biomass from the fermentation broth;
b) a concentration unit for concentrating the salt of the carboxylic acid in the low-biomass fermentation broth arranged downstream of the separation unit;
c) an acidification unit for acidifying the concentrated solution arranged downstream of the concentration unit; and
d) optionally a purification unit for purifying the salt of the carboxylic acid present in the low-biomass fermentation broth arranged between the separation unit and concentration unit.

The separation unit is preferably configured for carrying out a centrifugation, a separation, a precoat filtration, a microfiltration and/or an ultrafiltration.

The concentration unit is preferably configured for carrying out a nanofiltration, a reverse osmosis, a high-pressure reverse osmosis and/or a membrane distillation.

The acidification unit is preferably a settling vessel having a conical bottom and a discharge device arranged in the tip of the cone. It ideally directly adjoins the concentration unit.

The purification unit is preferably configured for carrying out a nanofiltration, a cation exchange, an anion exchange and/or an activated carbon purification. It is ideally arranged directly upstream of the concentration unit.

In a preferred embodiment of the invention, the plant further comprises a cooling crystallizer which is arranged downstream of the acidification unit and preferably directly adjoins the acidification unit.

In a preferred embodiment of the plant, the concentration unit consists of a two-stage reverse osmosis unit, with the first stage being connected to the second stage of the reverse osmosis unit via a permeate stream conduit for transferring the permeate stream from the first stage into the second stage.

The cooling crystallization unit of the apparatus of the invention can preferably have two stages, for example a cooling crystallization unit having a separate coolant system, with the second stage being connected to the first stage via at least one return conduit for the mother liquor to the coolant system of the first stage and the coolant system of the second stage having a separate feed conduit for a coolant.

The cooling crystallization unit consists of a contact crystallizer in a preferred embodiment.

### BRIEF DESCRIPTION OF THE DRAWINGS

The invention is illustrated below with the aid of drawings. In detail, the drawings show:

FIG. 1 a preferred embodiment of the invention and

FIGS. 2 (2.1 and 2.2) a further preferred embodiment of the invention.

### DESCRIPTION OF THE PREFERRED EMBODIMENTS

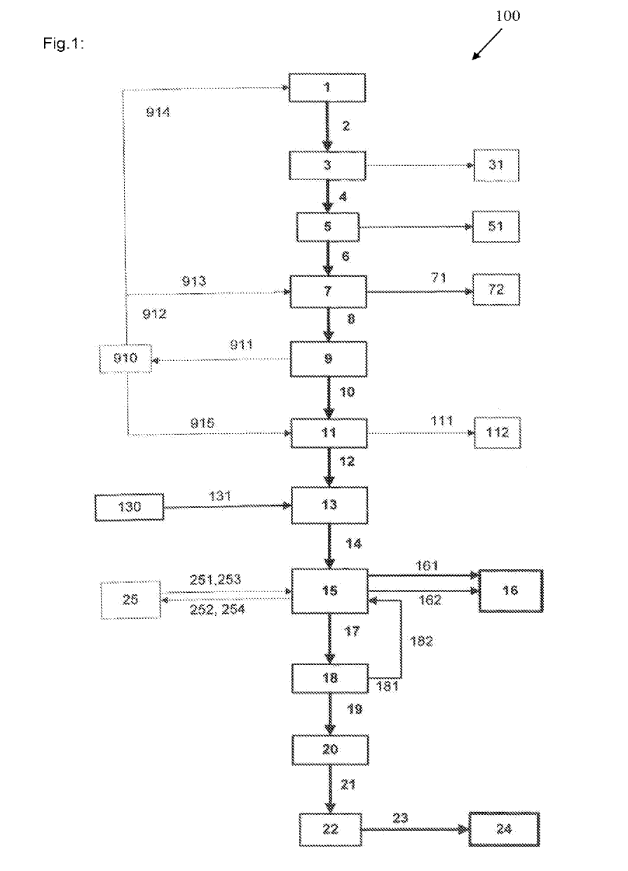

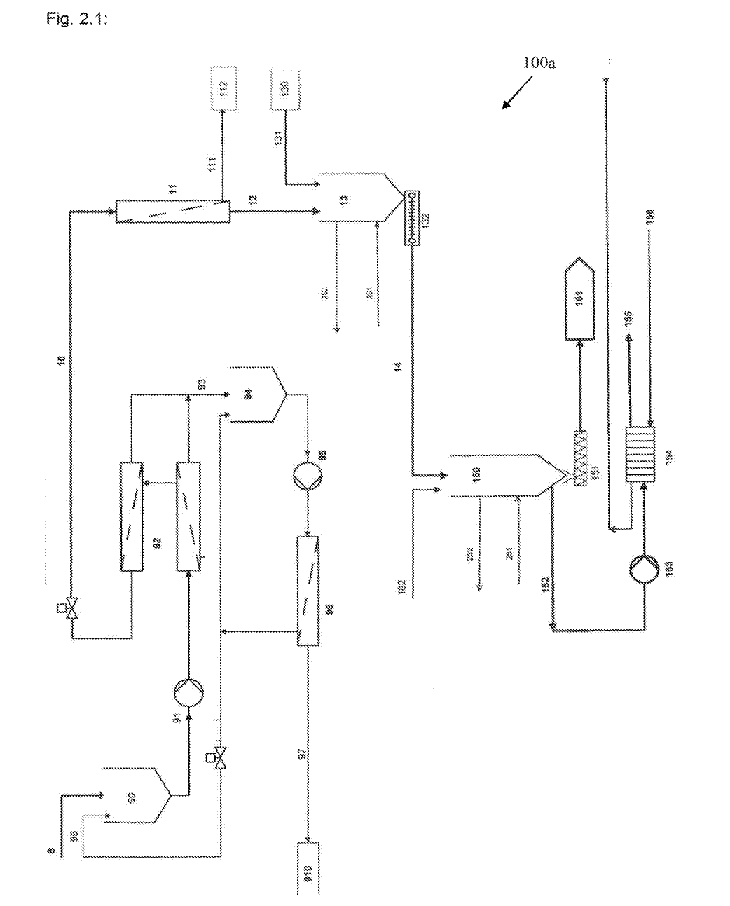

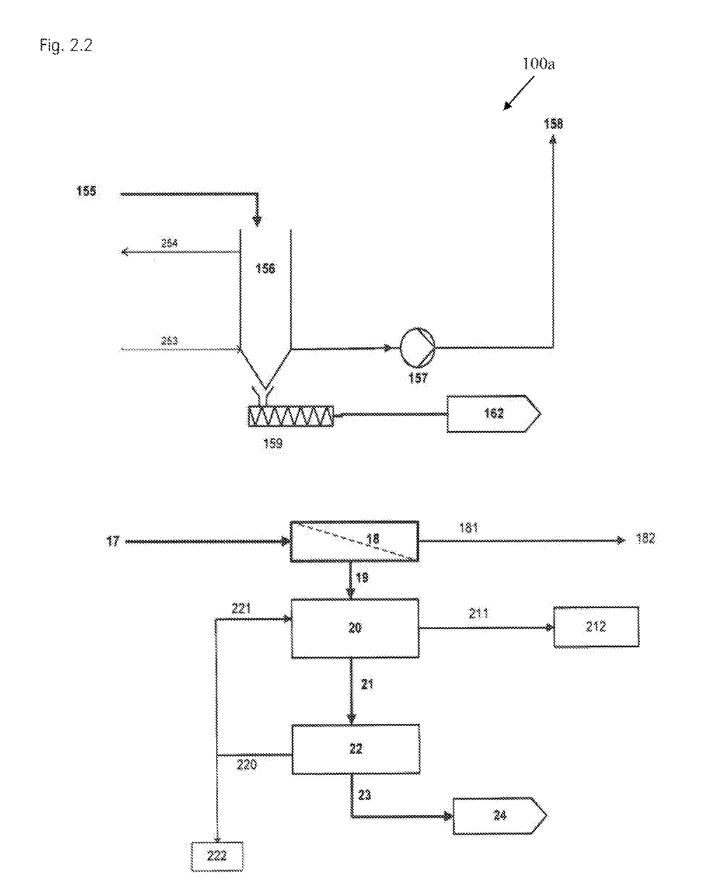

FIGS. 1 and 2 show inventive embodiments of the invention. To describe the figures, reference will be made in the following to the features of a plant equipped for carrying out the process of the invention.

According to FIG. 1, the plant 100 comprises a fermentation unit 1 in which a carboxylic acid is prepared by fermentation of carbohydrate-containing substrates by means of microorganisms. In particular, a fermentation broth which preferably contains a carboxylic acid salt and further impurities such as organic acids, by-products of the fermentation, microorganisms and constituents thereof and also residues of the substrates, e.g. sugar, is produced in the fermentation unit 1. The fermentation broth produced in the fermentation unit 1 is fed via a connecting piece 2 to a separator unit 3 and a preferably single-stage or multistage ultrafiltration unit 5 connected thereto via a connecting piece 4. In the separator unit, the biomass suspension 31 is separated from the fermentation broth so as to give a biomass-free fermentation broth. The biomass suspension 31 comprises essentially the microorganisms which have been separated off and remaining solids from the fermenter output. In the separator unit 3, the biomass 31 is precipitated by means of centrifugal force. The clear runnings from the separator are fed via the connecting piece 4 to the ultrafiltration unit 5. In the ultrafiltration unit 5, the fermentation broth coming from the separator unit 3 is purified in a second step. The membranes of the ultrafiltration unit 5 preferably have a separation limit of 0 kDa. The biomass-free fermentation broth is then fed via a connecting piece 6 to a purification unit 7. In the purification unit 7, the biomass-free fermentation broth is subjected to polishing by means of nanofiltration. Here, a nanofiltration membrane having a separation limit of from 100 to 400 Da is used. The process is carried out in such a way that the retentate 72 makes up not more than 2% of the total throughput. The retentate is discharged from the nanofiltration via the connecting piece 71. The permeate is subsequently fed via a connecting piece 8 to a concentration unit 9. Here, the content of the carboxylic acid salt in the biomass-free fermentation broth which has been purified by means of nanofiltration is concentrated to a value in the range from 7 to 50% by weight. Preference is given here to using a two-stage reverse osmosis configured as high-pressure reverse osmosis (pressure range up to 140 bar). The permeate 910 leaving the concentration unit 9 via the connecting piece 911 can be recirculated in the process. For example, it can be fed via connecting pieces 911, 912 as diafiltration water 913 to the purification unit 7 and/or via the connecting pieces 914 as make-up water for preparation of the media for the fermentation in the fermentation unit 1.

The concentrate from the concentration unit is fed via the connecting piece 10 directly to a further purification unit 11, so that no additional increase in pressure and no pump is necessary for this nanofiltration stage.

The nanofiltration arranged downstream of the reverse osmosis serves to concentrate the ammonium salt further when the osmotic pressure for the reverse osmosis becomes too high. Since part of the salt goes into the permeate in the nanofiltration, the solution can be concentrated still further. However, the permeate should then be recirculated upstream of the reverse osmosis.

The permeate from this second purification stage is recirculated to upstream of the reverse osmosis stage. The concentrated carboxylic acid salt solution is subsequently fed via a connecting piece 12 directly to an acidification unit 13 in which the carboxylic acid salt solution is admixed with a mineral acid, so that part of a carboxylic acid precipitates and a mixture of carboxylic acid solution and inorganic salt solution is obtained. The mineral acid is here fed from an acid unit 130 via a connecting piece 131 to the acidification unit 13. The introduction and discharge of cooling water into/from the integrated heat exchanger (not shown) on the acidification vessel occurs via the connecting pieces 251, 252. The acidification unit 13 is preferably configured as settling vessel having a conical bottom. The acidified carboxylic acid solution/salt solution mixture is fed via a connecting piece 14 to a cooling crystallization unit 15 in which the part of the carboxylic acid remaining in solution is isolated. The cooling crystallization unit 15 is preferably a multistage contact crystallizer. In the cooling crystallization unit 15, the cooling and crystallization of the carboxylic acid is effected by means of cooling brine 25 down to a crystallization temperature of from 0 to 10° C., with the cooling brine being fed and discharged into/from the crystallization unit 15 via connecting pieces 251, 252. A carboxylic acid crystal slurry can then be fed via a discharge device 161 and a connecting piece or a crystallizer 162 to a crystal purification 16. The discharge stream from the cooling crystallization (mother liquor) is fed via a connecting piece 17 to a work-up unit 18 in which this mother liquor is worked up, for example by means of a nanofiltration, so as to give a purified mother liquor stream and a carboxylic acid solution stream. The carboxylic acid solution stream 181 is fed back to the crystallization unit 15 via a connecting piece 182. The purified mother liquor stream, which now contains essentially only the inorganic salt solution, is fed via a connecting piece 19 to a thermal concentration unit 20, in which a multistage evaporation with vapor recirculation preferably takes place, and subsequently via a connecting piece 21 to a crystallization unit 22 in order to isolate the salt present in the purified mother liquor stream from the solution. A salt crystal slurry is then discharged via a connecting piece 23 to the salt crystallization 24.

According to FIG. 2, the feed stream 8 is fed into a reverse osmosis apparatus 9 which consists of two reverse osmosis stages 92, 96. The feed stream firstly goes into a circulation vessel 90 from where it is conveyed by means of a circulation pump 91 into the first stage of the reverse osmosis 92. The permeate from the first stage is conveyed via a connecting piece 93 into the circulation vessel 94 of the second stage 96. The concentrate stream is conveyed via a connecting piece 10 into the subsequent purification unit 11. This purification unit is a nanofiltration and is connected to the preceding reverse osmosis in such a way that no additional energy for increasing the pressure in the nanofiltration is necessary.

The permeate from the nanofiltration II is transferred via the connecting piece 12 into the acidification vessel 13.

The retentate 112 is removed from the plant 100, 100a via the connecting piece 111. It is proposed that the retentates from the two purification units be passed to utilization in terms of material in the form of an esterification by means of alcohol. In this way, there are no losses resulting from the two purification units.

The second stage of the reverse osmosis is operated using the process pump 95. While the permeate from the second stage goes via the connecting piece 97 to be used as make-up water for the fermentation 910, the concentrate goes via the connecting piece 98 to the circulation vessel 90.

In the acidification unit 13, acidification to the desired pH (in the example, succinic acid to pH 2.0) is effected by addition of acid 130 via the connecting piece 131. Cooling water is introduced and discharged via the connecting pieces 251 and 252. The acidified medium 14 is taken off via a discharge device 132 and fed into the first cooling crystallizer 150. The crystal slurry 161 is taken off via the discharge device 151 and passed to crystal work-up 16.

The solution 152 flowing out from the first cooling crystallizer 150 (“mother liquor”) is conveyed with the aid of the pump 153 via the heat exchanger 154 and via the connecting piece 155 into the second cooling crystallizer 156. In the heat exchanger 154, regenerative heat exchange between cold mother liquor 158 from the second crystallizer and the hotter feed stream 152 to the second crystallizer 156 via the connecting piece 155 takes place.

The second cooling crystallizer 156 is operated using cooling brine 253; 254. The crystal slurry 162 is taken off via a discharge device 159 and is likewise passed to the treatment of the carboxylic acid crystals 16.

The mother liquor 17 flowing out from the heat exchanger 154 is conveyed into the third purification unit 18 for further work-up. The residual carboxylic acid solution 182 present in the permeate is recirculated via the connecting piece 181 into the first crystallizer 150, while the concentrate goes via the connecting piece 19 to the multistage thermal concentration of the salt solution 20. The vapor 212 arising there is recirculated via the connecting piece 211 to thermal utilization and utilization in terms of material in the production plant. In one embodiment, it is proposed that this vapor be utilized thermally in the concentration unit 9 when this unit comprises a membrane distillation. For utilization of this vapor in terms of material, it can be used for flushing and cleaning purposes, in particular in the fermentation unit 1.

The salt concentrate comes into the evaporative crystallization 22 via a connecting piece 21. The finished salt crystals 24 (in the example, ammonium sulfate) are taken off via a connecting piece 23. The salt mother liquor 220 formed is recirculated partly to the evaporation 20, while another part thereof is discharged into the wastewater 222.

### EXAMPLE

A fermentation broth containing ammonium succinate was prepurified by separation and ultrafiltration. A nanofiltration having a separation limit of 200 Da was subsequently carried out. The permeate from the nanofiltration had a succinate content of 68.5 g/l.

The concentration was increased to 212 g/l by means of high-pressure reverse osmosis. The concentration factor was 3.1.

1 kg of this solution having a pH of 6.3 was acidified to a pH of 2.45 by means of concentrated sulfuric acid while cooling. The acid consumption was 102 ml of 96% strength sulfuric acid. The solution was subsequently cooled. A moist crystal slurry having a mass of 480 g was then separated off. The color of the crystal slurry was light, with a slightly yellowish color. The supernatant solution amounted to 708 g. At a temperature of 8° C., 20.5 g/l of succinic acid and 350 g/l of sulfate as ammonium sulfate were still present.

### LIST OF REFERENCE NUMERALS

1 Fermentation unit
2 Connecting piece
3 Separator unit
4 Connecting piece
5 Ultrafiltration unit
6 Connecting piece
7 Purification unit 1
8 Connecting piece
9 Concentration unit or membrane process
10 Connecting piece
11 Purification unit 2
12 Connecting piece
13 Acidification unit
14 Connecting piece
15 Cooling crystallization
16 Crystallization unit
17 Connecting piece
18 Purification unit 3
19 Connecting piece
20 Thermal concentration unit
21 Connecting piece
22 Evaporative crystallization
23 Connecting piece
24 Salt crystallization
25 Cooling water/Cooling brine unit
31 Biomass suspension
51 Retentate from ultrafiltration
71 Connecting piece
72 Retentate from nanofiltration
90 Circulation vessel
91 Circulation pump
92 1st stage of reverse osmosis
93 Connecting piece
94 Circulation vessel, 2nd stage
95 Circulation pump
96 2nd stage of reverse osmosis
97 Permeate, 2nd stage
98 Concentrate, 2nd stage
100, 100a Plant
111 Connecting piece
112 Retentate from nanofiltration 2
130 Acid unit
131 Connecting piece
132 Discharge device
150 Cooling crystallizer 1 or cooling crystallizer
151 Discharge device
152 Connecting piece
153 Pump
154 Heat exchanger
155 Connecting piece
156 Cooling crystallizer 2 or cooling crystallizer
157 Pump
158 Cold mother liquor
159 Discharge device
161 Crystal slurry from crystallizer 1
162 Crystal slurry from crystallizer 2
181 Connecting piece
182 Product recirculation
211 Connecting piece
212 Vapor
220 Connecting piece
221 Mother liquor recirculation
222 Wastewater
251 Cooling water, forward flow
252 Cooling water, backflow
253 Cooling brine, forward flow
254 Cooling brine, backflow
910 Permeate from reverse osmosis
911 Connecting piece
912 Connecting piece
913 Water for diafiltration
914 Make-up water for fermentation
915 Water for diafiltration

1.-16. (canceled)

17. A process for isolating a carboxylic acid from a fermentation broth, the process comprising:
separating a biomass from the fermentation broth containing a salt of the carboxylic acid to produce a low-biomass solution;
concentrating the salt of the carboxylic acid in the low-biomass solution to form a concentrated solution;
acidifying the concentrated solution; and
precipitating the carboxylic acid obtained by acidification.

18. The process of claim 17 wherein the separation of the biomass is performed in a first step by centrifugation, separation, precoat filtration, or microfiltration, and in a second step by ultrafiltration.

19. The process of claim 17 wherein prior to the acidification of the concentrated solution, the process comprises purifying the low-biomass solution or the concentrated solution by at least one of nanofiltration, cation exchange, anion exchange, or activated carbon purification.

20. The process of claim 17 wherein a concentration of the concentrated solution is above a solubility concentration of the carboxylic acid.

21. The process of claim 17 wherein the salt of the carboxylic acid is concentrated from 1%-10% by weight to 40%-50% by weight.

22. The process of claim 17 wherein the salt of the carboxylic acid is concentrated from 3%-7% by weight to 20%-25% by weight.

23. The process of claim 17 wherein concentrating the salt of the carboxylic acid in the low-biomass solution comprises a membrane process performed in one, two, or more stages.

24. The process of claim 23 wherein the membrane process is at least one of nanofiltration, reverse osmosis, high-pressure reverse osmosis, or membrane distillation.

25. The process of claim 17 wherein
a pH of the concentrated solution after the concentration is between 5.0 and 8.0; or
a pH in the concentrated solution after the acidification is between 1.8 and 3.0.

26. The process of claim 17 wherein the precipitation of the carboxylic acid is performed by way of a crystallization comprising cooling crystallization and isolation of the crystallized carboxylic acid.

27. The process of claim 26 wherein a mother liquor remaining after isolation of the crystallized carboxylic acid is, after a concentration of the carboxylic acid present therein has been increased, fed to the cooling crystallization.

28. The process of claim 17 wherein concentrating the salt of the carboxylic acid is performed with at least partial recovery of energy consumed by concentrating the salt of the carboxylic acid.

29. The process of claim 17 wherein prior to the acidification of the concentrated solution, the process comprises purifying the low-biomass solution or the concentrated solution, wherein the concentration or the purification is performed with at least partial recovery of energy consumed by the concentration or a fine purification step.

30. The process of claim 17 comprising feeding discharge streams obtained in the concentration to an upstream portion of the process or to an esterification.

31. The process of claim 17 wherein
the carboxylic acid has a solubility at 20° C. of 5 to 110 g/l;
the carboxylic acid is at least one of fumaric acid, succinic acid, adipic acid, itaconic acid, threonine, methionine, aspartic acid, glutamic acid, oxalic acid, asparagine, glutamine, histidine, isoleucine, leucine, phenylalanine, tryptophan, tyrosine, or valine; or
the salt of the carboxylic acid in the concentrated solution has a two-fold or five-fold greater solubility than the carboxylic acid.

32. A plant for isolating a carboxylic acid from a fermentation broth containing salt of the carboxylic acid, the plant comprising:
a separation unit for separating biomass from the fermentation broth;
a concentration unit downstream of the separation unit for concentrating the salt of the carboxylic acid in a low-biomass fermentation broth to form a concentrated solution; and
an acidification unit downstream of the concentration unit for acidifying the concentrated solution.

33. The plant of claim 32 wherein
the separation unit is configured for performing a centrifugation, a separation, a precoat filtration, a microfiltration, or an ultrafiltration;
the concentration unit is configured for performing a nanofiltration, a reverse osmosis, a high-pressure reverse osmosis, or a membrane distillation;
the acidification unit is a settling vessel having a conical bottom and a discharge device disposed in a tip of a cone of the settling vessel; or
the plant comprises a purification unit disposed between the separation unit and the concentration unit for purifying the salt of the carboxylic acid in the low-biomass fermentation broth, wherein the purification unit is configured for performing a nanofiltration, a cation exchange, an anion exchange, or an activated carbon purification.

34. The plant of claim 32 comprising a cooling crystallizer disposed downstream of the acidification unit.
